(19) United States
(12) Patent Application Publication
(10) Pub. No.: US 2004/0122081 A1
(43) Pub. Date: Jun. 24, 2004

(54) Pharmaceutical compositions and methods of using taxane derivatives

(76) Inventors: Uday Shankar Gogate, North Brunswick, NJ (US); Robert Kevin Perrone, Belle Mead, NJ (US); Krishnaswamy Srinivas Raghavan, Cranbury, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/704,165

(22) Filed: Nov. 7, 2003

Related U.S. Application Data
(60) Provisional application No. 60/424,921, filed on Nov. 8, 2002.

(51) Int. Cl. A61K 31/337

(52) U.S. Cl. 514/449

(57) ABSTRACT

The present invention relates to a novel intravenous formulation for a taxane chemotherapeutic agent. The agent is formulated as a two-container, i.e. vial system, with one container holding the therapeutic agent in a solvent with a buffer and the other container holding a co-solvent in a buffer. The contents of the two containers are mixed prior to administration.

This application claims priority benefit under Title 35 § 119(e) of U.S. provisional Application No. 60/424,921 filed Nov. 8, 2002.

### FIELD OF THE INVENTION

The present invention relates to a novel two-container formulation for taxane compounds, said formulation characterized by increased solubility and stability, and resistance to oxidation.

### BACKGROUND OF THE INVENTION

U.S. Pat. No. 5,646,176 discloses taxane derivatives and their use as anti-tumor agents. The compounds disclosed herein have been found useful for the treatment of certain types of cancer including bladder and gastric cancer.

Taxol® (paclitaxel) is a natural product extracted from the bark of Pacific yew trees, Taxus brevifolia. It has been shown to have excellent antitumor activity in in vivo animal models, and recent studies have elucidated its unique mode of action, which involves abnormal polymerization of tubulin and disruption of mitosis. It has recently been approved for the treatment of refractory advanced ovarian cancer and breast cancer; and studies involving other cancers have shown promising results. The results of paclitaxel clinical studies are reviewed by numerous authors, such as by Rowinsky and Donehower in “The Clinical Pharmacology and Use of Antimicrotubule Agents in Cancer Chemotherapeutics”, Pharmac. Ther., 52:35-84, 1991; by Spencer and Faulds in “Paclitaxel, A Review of its Pharmacodynamic and Pharmacokinetic Properties and Therapeutic Potential in the Treatment of Cancer”, Drugs, 48 (5) 794-847, 1994; by K. C. Nicolaou et al. in “Chemistry and Biology of Taxol”, Angew. Chem., Int. Ed.Engl., 33: 15-44, 1994; by F. A. Holmes, A. P. Kudelka, J. J. Kavanaugh, M. H. Huber, J. A. Ajani, V. Valero in the book “Taxane Anticancer Agents Basic Science and Current Status” edited by Gunda I. Georg, Thomas T. Chen, Iwao Ojima, and Dolotrai M. Vyas, 1995, American Chemical Society, Washington, D.C., 31-57; by Susan G. Arbuck and Barbara Blaylock in the book “TAXOL® Science and Applications” edited by Mathew Suffness, 1995, CRC Press Inc., Boca Raton, Fla., 379-416; and also in the references cited therein.

Derivatives of Taxol® have been found to possess antitumor activity; however, it has been challenging to prepare formulations of these derivatives because of their inherent insolubility and their susceptibility to oxidation when used with standard formulations of Taxol® containing polyoxyethylated (POE) castor oil and other carriers.

### SUMMARY OF THE INVENTION

The present invention is directed to a novel two container formulation which comprises, in one container

at least one taxane compound of the formula

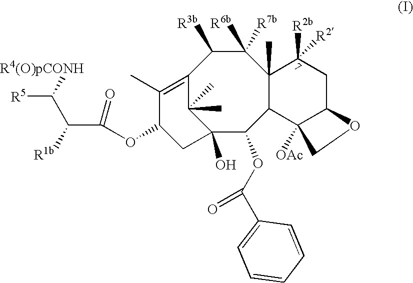

wherein:

R^{1b} is hydroxy, protected hydroxy, —OCH₂SCH₃, —OC(O)R^{x} or —OC(O)OR^{x};

R² is hydrogen, and

R^{2b} is hydrogen, hydroxy, protected hydroxy, —OCH₂SCH₃ or —OC(O)OR^{x};

R^{3b} is hydrogen, hydroxy, protected hydroxy, C₁₋₆ alkyloxy, —OC(O)R^{x}, —OCH₂SCH₃ or —OC(O)OR^{x};

one of R^{6b} or R^{7b} is hydrogen and the other is hydroxy, protected hydroxy, C₁₋₆ alkanoyloxy or —OCH₂SCH₃; or

R^{6b} and R^{7b} together form an oxo group; with the proviso that at least one of R^{1b}, R^{2b}, R^{3b}, R^{6b} or R^{7b} is —OCH₂SCH₃;

p is 0 or 1;

R^{x} is a radical of the formula

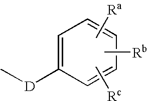

wherein

D is a bond or C₁₋₆ alkyl; and

R^{a}, R^{b} and R^{c} are independently hydrogen, amino C₁₋₆ alkylamino, di-C₁₋₆ alkylamino, halogen, C₁₋₆ alkyl, or C₁₋₆ alkoxy;

R⁴ and R⁵ are independently C₁₋₆ alkyl, C₂₋₆ alkenyl, C₂₋₆alkynyl, or —ZR⁶; wherein Z is a direct bond, C₁₋₆ alkyl or C₂₋₆ alkenyl; and

R⁶ is aryl, substituted aryl, C₃₋₆ cycloalkyl, or heteroaryl;

b) in a suitable solvent; and

c) a pharmaceutically effective amount of a buffer;

and in a second container;

d) a pharmaceutically effective amount of a co-solvent; and

e) a pharmaceutically effective amount of a buffer.

The contents of the two containers are mixed prior to administration.

In a preferred embodiment, the formulation of the invention employs a compound of the formula

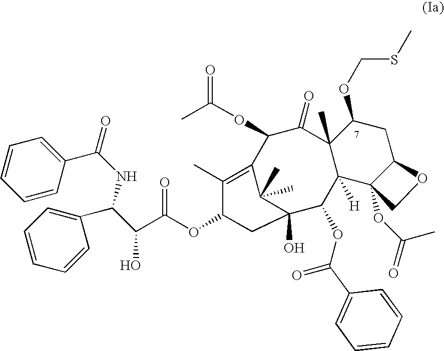

with the above described substituents.

The compounds represented by formula (I) are novel compounds that are useful in the treatment of a variety of cancers and other abnormal proliferative diseases. The novel formulation increases the solubility of the insoluble compounds and provides for a two-container formulation; one containing the compound in solution and the other containing the appropriate diluent for administration of the compound.

The invention also provides methods for their use in the treatment of cancer.

### DETAILED DESCRIPTION OF THE INVENTION

The present invention is directed to a novel two-container formulation which comprises, in one container

at least one taxane compound of the formula

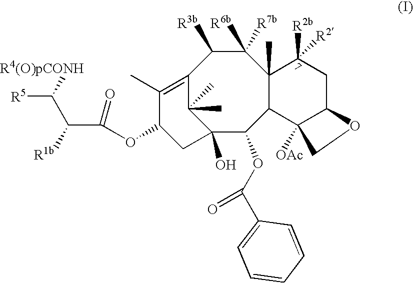

wherein:

R^{1b} is hydroxy, protected hydroxy, —OCH₂SCH₃, —OC(O)R^{x} or —OC(O)OR^{x};

R² is hydrogen, and

R^{2b} is hydrogen, hydroxy, protected hydroxy, —OCH₂SCH₃ or —OC(O)OR^{x};

R^{3b} is hydrogen, hydroxy, protected hydroxy, C₁₋₆alkyloxy, —OC(O)R^{x}, —OCH₂SCH₃ or —OC(O)OR^{x};

one of R^{6b} or R^{7b} is hydrogen and the other is hydroxy, protected hydroxy, C₁₋₆ alkanoyloxy or —OCH₂SCH₃; or

R^{6b} and R^{7b} together form an oxo group; with the proviso that at least one of R^{1b}, R^{2b}, R^{3b}, R^{6b} or R^{7b} is —OCH₂SCH₃;

p is 0 or 1;

R^{x} is a radical of the formula

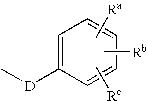

wherein

D is a bond or C₁₋₆ alkyl; and

R^{a}, R^{b} and R^{c} are independently hydrogen, amino C₁₋₆ alkylamino, di-C₁₋₆ alkylamino, halogen, C₁₋₆ alkyl, or C₁₋₆ alkoxy;

R⁴ and R⁵ are independently C₁₋₆ alkyl, C₂₋₆ alkenyl, C₂₋₆alkynyl, or —ZR⁶; wherein Z is a direct bond, C₁₋₆ alkyl or C₂₋₆ alkenyl; and

R⁶ is aryl, substituted aryl, C₃₋₆ cycloalkyl, or heteroaryl;

b) in a suitable solvent; and

c) a pharmaceutically effective amount of a buffer;

and in a second container;

d) a pharmaceutically effective amount of a co-solvent; and

e) a pharmaceutically effective amount of a buffer; to provide drug stability after the contents of the two containers are mixed.

In a preferred embodiment, the compound of formula I is the compound of formula Ia shown below, which is 7-O-methylthiomethylpaclitaxel

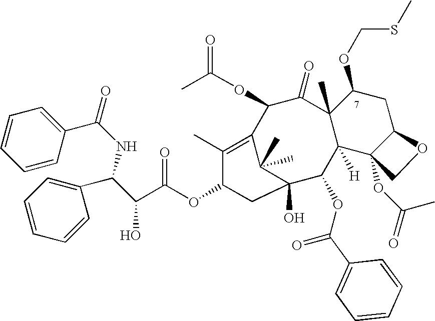

with the above described substituents.

Listed below are definitions of various terms used to describe this invention. These definitions apply to the terms as they are used throughout this specification, unless otherwise indicated in specific instances.

“Alkyl” means a straight or branched saturated carbon chain having from one to six carbon atoms; examples include methyl, ethyl, n-propyl, isopropyl, n-butyl, sec-butyl, isobutyl, t-butyl, n-pentyl, sec-pentyl, isopentyl, and n-hexyl.

“Alkenyl” means a straight or branched carbon chain having at least one carbon-carbon double bond, and having from two to six carbon atoms; examples include ethenyl, propenyl, isopropenyl, butenyl, isobutenyl, pentenyl, and hexenyl.

“Alkynyl” means a straight or branched carbon chain having at least one carbon-carbon triple bond, and from two to six carbon atoms; examples include ethynyl, propynyl, butynyl, and hexynyl.

“Aryl” means aromatic hydrocarbon having from six to ten carbon atoms; examples include phenyl and naphthyl. “Substituted aryl” means aryl substituted with at least one group selected from C₁₋₆ alkanoyloxy, hydroxy, halogen, C₁₋₆ alkyl, trifluoromethyl, C₁₋₆ alkoxy, aryl, C₂₋₆ alkenyl, C₁₋₆ alkanoyl, nitro, amino, and amido.

“Halogen” means fluorine, chlorine, bromine, and iodine.

“Taxane derivative” refers to a compound having a taxane moiety bearing a C₁₃ sidechain.

“Heteroaryl” means a five- or six-membered aromatic ring containing at least one and up to four non-carbon atoms selected from oxygen, sulfur and nitrogen. Examples of heteroaryl include thienyl, furyl, pyrrolyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, and like rings.

“Hydroxy protecting groups” include, but is not limited to, ethers such as methyl, t-butyl, benzyl,p-methoxybenzyl, p-nitrobenzyl, allyl, trityl, methoxymethyl, methoxyethoxymethyl, ethoxyethyl, tetrahydropyranyl, tetrahydrothiopyranyl, and trialkylsilyl ethers such as trimethylsilyl ether, triethylsilyl ether, and t-butyldimethylsilyl ether; esters such as benzoyl, acetyl, phenylacetyl, formyl, mono-, di-, and trihaloacetyl such as chloroacetyl, dichloroacetyl, trichloroacetyl, trifluoroacetyl; and carbonates such as methyl, ethyl, 2,2,2-trichloroethyl, allyl, benzyl, and p-nitrophenyl.

Additional examples of hydroxy protecting groups may be found in standard reference works such as Greene and Wuts, Protective Groups in Organic Synthesis, 2d Ed., 1991, John Wiley & Sons, and McOmie, Protective Groups in Organic Chemistry, 1975, Plenum Press. Methods for introducing and removing protecting groups are also found in such textbooks.

The term “container” means any pharmaceutically acceptable vessel that could be used to hold a liquid solution and that is amenable to the administration of an intravenous or intramuscular formulation. These include vials, sterile bags, syringes and the like.

The formulation of the present invention provides an advantageous method for the administration of the compound by increasing the solubility, decreasing the oxidation of and maintaining drug stability during shelf-life storage and following aqueous dilution.

The compounds of the invention are microtubule-stabilizing agents and, thus, can be used to treat a variety of cancers or other diseases of abnormal cell proliferation. The methods of the invention are particularly useful for administering the compounds of the invention to a patient suffering from cancer or other hyperproliferative cellular disease. As used herein, the term “cancer” includes, but is not limited to, solid tumors and blood born tumors. The term cancer refers to disease of skin, tissues, organs, bone, cartilage, blood and vessels. The term “cancer” further encompasses primary and metastatic cancers.

The compositions of the invention are preferably provided in the form of unit doses in sealed vials, preferably glass vials, most preferably Type I glass vials closed with elastomer stoppers.

Compound Ia by itself has low intrinsic aqueous solubility (<0.1 μg/ml) and a salt formation could not be used since the compound does not ionize in a desirable physiological pH range. Therefore, it was necessary to formulate the compound in such a way to get the desired solubility at a physiological pH and maintain stability prior to administration. It was determined that while the solubility is higher in solvents other than water, drug precipitation occurs upon aqueous dilution.

Studies were undertaken to develop a formulation that included a 60 mg/vial, 15 mg/mL of compound along with 37.6 mL per vial of diluent. Both substituents are buffered in order to achieve optimum stability.

It is expected that the daily human dose is approximately 120 mg. In order to achieve a practical volume of infusion, a solution with higher drug concentration (than 0.1 μg/ml aqueous solubility) is required.

Various co-solvents were evaluated. Preferred solvents of the invention include ethanol, t-butyl alcohol, propylene glycol, glycerin, benzyl benzoate and N,N-dimethylacetamide. Particularly preferred are ethanol and t-butyl alcohol and these were further studied. The drug solubility was evaluated as a function of dehydrated alcohol or tertiary butyl alcohol concentration. It was discovered that 75% v/v ethanol (dehydrated alcohol) in water for injection provided the highest solubility of the preferred compounds at >17.5 mg/mL. A drug concentration of 15 mg of compound/mL in the 75% v/v ethanol:water was selected for further study.

It was also determined that the first container should include a buffer to help stability. Preferred buffering agents include citrate, tartrate, succinate, fumarate, oxalate, benzoate, acetate or lactate buffers, with the tartrate particularly preferred.

The 15 mg/mL solution including a 10 mM tartrate buffer which provided adequate solubility and stability, however, the solution could not be injected directly into patients because the non-aqueous components amounted to greater than 20% which potentially causes irritation at the injection site. Further dilution of this solution is therefore required.

Various diluents such as sodium chloride injection and dextrose injection were tried but both resulted in drug precipitation. Polysorbate, polyethylene glycol and polyoxyethylated (POE) castor oil are the preferred co-solvents with POE castor oil particularly preferred. The use of polyoxyethylated castor oil (Cremophor EL/BASF) was further evaluated. Drug solubility in three solutions containing mixtures of POE castor oil with ethanol was studied. An aqueous solution containing 7.5% ethanol and 4% POE castor oil was selected.

While this solution solves the injection site irritation problem, it was noted that drug degradation occurs due to peroxide impurities in the POE castor oil. It was determined that the degradation pathway could be avoided by separating the drug substance solution from the POE castor oil by utilizing a two-container system.

A preferred composition contains in the first container about 1 μg/mL to about 20 mg/mL of Compound I, about 5% to about 95% v/v ethanol (0.05 to 0.95 mL/mL) in an aqueous tartrate buffer, and in the second vial about 1% to about 95% v/v (0.01 to 0.95 mL/mL) of a polyoxyethylated castor oil in an aqueous tartrate buffer.

A particularly preferred composition is detailed below in Tables VII and VIII.

The compositions of the invention are preferably provided in the form of unit doses in sealed vials, preferably glass vials, most preferably Type I glass vials closed with elastomer stoppers. The preferred unit dose will contain a pharmaceutically effective amount of a taxane derivative, together with a buffer and solvent in one vial and the buffered POE castor oil in the second vial.

By way of illustration, and without serving as limitations in any way, the following examples serve to illustrate the practice of the invention.

### EXAMPLES

The compound was subjected to early solubility studies, to determine which co-solvent could be used to increase drug solubility, according to the following procedure.

### Example 1

### Solubility of Compound in a Co-Solvent:Water Mixture

Approximately 25 mg of drug substance was added to 2 mL aqueous solution of ethanol (33%, 50% and 75% v/v). An additional 10 mg of drug substance was added to the 75% sample as all drug appeared to dissolve. A similar study was performed by adding approximately 25 mg drug substance to 2 mL aqueous solution of tertiary butyl alcohol (33%, 50% and 66% v/v). Samples were stirred for over 16 hours, filtered through 0.45μ nylon syringe filters, diluted and analyzed by HPLC for drug concentration. The results shown in Table I indicate that among the conditions evaluated, 75% v/v dehydrated alcohol in water for injection provided the highest solubility. Based on the results of these studies, a formulation of 15 mg/mL drug substance in 75% v/v ethanol:water was selected for further studies.

TABLE I

| Vehicle | | Solubility |
|---|---|---|
| Co-Solvent | % v/v | (mg/mL) |
| Tertiary Butyl Alcohol | 33 | 0.19 |
| | 50 | 3.13 |
| | 66 | 12.95 |
| Dehydrated Alcohol, USP | 33 | 0.03 |
| | 50 | 1.64 |
| | 75 | >17.5 |

The effect of pH on the drug substance stability was also studied. The buffer pH providing maximum stability was determined by comparing the stability of prototype formulations of the drug substance. Initial experiments evaluated solutions containing 0.2 mg drug/mL in 16.7% v/v ethanol:0.1M citrate buffers. Relative area percents of drug peaks were evaluated following 2 days storage at 85° C. HPLC analysis demonstrated that the best stability was achieved at buffer 4.5. Subsequent experiments evaluated stability (1 mg drug/mL) in 75% v/v ethanol:0.01M tartrate buffers. Three mL aliquots of samples were dispensed into 5 cc Type I glass vials and closed with West 4405/50 20 mm stoppers. Percent drug substance remaining, and impurities were evaluated following 18 days storage at 50° C. and compared to initial values. A solution with apparent pH 5.4 (corresponding to tartrate buffer pH 3.8), was observed to be most stable. Based on these results, tartrate buffer pH 3.8 was selected for further experiments because the pH of maximum stability is within the buffering range of tartaric acid (pKₐ₁=3.02, pKₐ₂=4.54).

TABLE II

| | | | Total |
|---|---|---|---|
| | | | Impurity |
| | | % Compound | Index |
| Buffer pH | Apparent pH | Remaining | (Area %) |
| 2.6 | 3.88 | 94.4 | 4.16 |
| 3.0 | 4.67 | 97.1 | 2.51 |
| 3.5 | 5.15 | 97.1 | 2.06 |
| 3.6 | 5.20 | 98.1 | 1.36 |
| 3.8 | 5.39 | 101 | 1.34 |
| 4.0 | 5.81 | 98.1 | 1.99 |
| 4.0 | 5.69 | 100 | 1.62 |
| 4.2 | 5.99 | 99 | 1.67 |
| 4.4 | 6.24 | 100 | 1.84 |

Drug solution (15 mg/mL, 75% ethanol/10 mM tartrate buffer, apparent pH 5.4) was found to provide adequate solubility and stability. However, this solution cannot be injected directly into patients as the non-aqueous components exceed 20%, thus potentially causing irritation at the injection site. Dilution of this solution with aqueous diluents such as 0.9% sodium chloride injection or 5% dextrose injection causes drug precipitation. It has been shown that the precipitation can be avoided by inclusion of a co-solvent such as polyoxyethylated (POE) castor oil in the formulation. Subsequently, solubility of the drug substance was determined in solutions containing various amounts of dehydrated alcohol and POE castor oil. Approximately 20 mg of drug was added to 3 mL aliquots of the solutions shown below in Table III. Samples were stirred for 16 hours, filtered trough 0.45 micron nylon syringe filters and analyzed by HPLC for drug concentration. Results in Table III indicate that an aqueous solution containing 7.5% dehydrated alcohol and 4% POE castor oil provides adequate drug solubility (>1.5 mg/mL) with a minimized amount of co-solvent.

TABLE III

| | |
|---|---|
| Aqueous Vehicle | Drug Solubility |

| Dehydrated Alcohol v/v | POE Castor Oil v/v | (mg/mL) |
|---|---|---|
| 9.375% | 5% | 2.91 |
| 7.50% | 4% | 2.38 |
| 3.75% | 2% | 0.96 |

Solubility of four lots of drug substance was evaluated in the prototype formulation developed; 75% v/v dehydrated alcohol, 10 mM tartrate buffer resulting in an apparent pH of 5.4. 15 mg/mL of drug substance were used. 5 mL aliquots of the solution were dispensed into glass vials and an additional (˜50 mg) of drug substance was added. The vials were closed, sealed and stirred for 16 hours at room temperature. Samples were filtered and analyzed by HPLC for drug concentration. The average solubility of the compound at room temperature was approximately 22 mg/mL for the four lots evaluated. Similar experiments were performed to determine the equilibrium solubility of the compound in the above formulation at 5±3° C. The solubility at 5±3° C. was observed to be 23 mg/mL and about the same as at 25±3° C.

The solubility of the compounds of the invention in dehydrated alcohol/polyoxyethylated castor oil systems at 24±3° C. was also evaluated. As shown in Table III, an aqueous solution containing 7.5% dehydrated alcohol and 4% POE castor oil provide adequate solubility (>1.5 mg/mL) with a minimized amount of co-solvent. However, as shown in Table IV, drug degradation occurs due to peroxide impurities in the POE castor oil.

TABLE IV

| | Potency | |
|---|---|---|
| Storage Conditions | (mg/mL) | Total Impurities |
| Initial | 2.2 | 2.1 |
| 6 days @ 50° C. | 2.0 | 10.9 |
| 16 days @ 50° C. | 1.9 | 16.5 |

The degradation pathway can be avoided by either separating the drug substance from POE castor oil via a two-container system as disclosed herein or by adding appropriate antioxidants, as disclosed in a related application.

Table V shows the effect of the presence of POE castor oil on the stability of the injection solution containing ethanol and pH 5.4 tartrate buffer. As shown below, the stability of the solution containing POE castor oil was much lower than the injection solution without the co-solvent.

TABLE V

| | | % potency |
|---|---|---|
| Solution | Days Stored at 50° C. | remaining |
| with POE castor oil 4% | 16 | 86 |
| w/o POE castor oil | 28 | 100 |

A study was also undertaken to determine the equilibrium solubility of Compound Ia in the mixture obtained by mixing the contents of the two vial formulation. Ten milliliters of the mixed solution was transferred into four 10 cc Type I flint glass vials. To each of these aliquots, approximately 25 mg of drug substance was added. A different lot of drug substance was added to each vial. Vials were closed with West Teflon-faced stoppers, sealed with aluminum seals and stirred for 16 hours at room temperature, protected from light. Samples were filtered through 0.45 micron hydrophilic PVDF membranes and analyzed by HPLC for drug concentration. Solubility results are listed below in Table VI. The average equilibrium solubility value obtained at room temperature (˜2.58 mg/mL) is well above the expected drug concentration of 1.5 mg/mL obtained after mixing the two vials. Separate studies were done to determine the equilibrium solubility of drug substance at 5±3° C. in the solution obtained after mixing the two vials. Samples were prepared and stirred at 24±3° C. for about 5 hours and placed in a 5±3° C. storage chamber for about 17 hours. Upon removal from the storage chamber, samples were immediately filtered and analyzed by HPLC for drug concentration. The resulting solubility was found to be 2.46 mg/mL showing that the solubility is not adversely affected by storage at 5±3° C.

TABLE VI
Equilibrium Solubility of Compound Ia in Diluted
Drug Product^{a}

| | Solubility |
|---|---|
| Temperature | (mg/mL) |
| 24 ± 3° C. | 2.57 |
| | 2.62 |
| | 2.56 |
| | 2.58 |
| Average @ 24 ± 3° C. | 2.58 |
| 5 ± 3° C. | 2.46 |

^{a}4.1 mL Drug Injection diluted with 36.7 mL of Diluent for Compound Ia results in final concentrations of 7.5% ethanol, 4% POE castor oil in tartrate buffer.

The quantitative composition for the two-vial formulation is shown below in Tables VII and VIII. A 10% overage of drug solution for vial-needle-syringe hold-up was added.

TABLE VII
Quantitative Composition of Active Injection,
60 mg/vial (15 mg/mL)

| | | Amount per | Amount per |
|---|---|---|---|
| Ingredient | Rationale for Use | mL | Vial |
| Compound Ia | Active | 15.0 mg | 66.0 mg |
| Dehydrated | Solvent | 0.75 mL | 3.30 mL |
| Alcohol, USP | | | |
| Tartaric Acid | Stabilizer (buffer) | 0.22 mg | 0.968 mg |
| NF/EP | | | |
| Sodium Tartrate | Stabilizer (buffer) | 0.31 mg | 1.364 mg |
| Dihydrate | | | |
| Water for Injection, | Solvent | q.s. to 1.0 mL | q.s. to 4.4 mL |
| USP | | | |

TABLE VIII
Quantitative Composition of Diluent for
Active Injection, 36.7 mL/vial

| | | Amount per | |
|---|---|---|---|
| Ingredient | Rationale for Use | mL | Amount per Vial |
| BMS Purified | Co-Solvent | 0.044 mL | 1.615 mL |
| Polyoxyethylated | | | |
| Castor Oil | | | |
| Tartaric Acid | Stabilizer (buffer) | 0.086 mg | 3.156 mg |
| NF/EP | | | |
| Sodium Tartrate | Stabilizer (buffer) | 2.07 mg | 75.97 mg |
| Dihydrate | | | |
| Water for | Solvent | q.s. to 1.0 mL | q.s. to 36.7^{a} mL, |
| Injection, USP | | | |

^{a}Includes 10% overage for vial-needle-syringe hold-up.

Further studies were conducted which indicate that the selected formulation is stable for at least 12 months when stored at 25° C. at a relative humidity of 60% when protected from light. The diluent was also observed to be stable for at least 12 months when stored at 25° C.

### Example 2

### Preparation of 7-O-methylthiomethylpaclitaxel (Compound Ia)

Benzoyl peroxide (0.98 g, 4 mmol) was added to a vigorously stirred mixture of paclitaxel (0.85 g, 1 mmol) and dimethyl sulfide (0.72 mL, 8 mmol) in dry acetonitrile (10 ml) at 0. degree. C. Stirring was continued for 2.5 hours at 0. degree. C. Progress of the reaction was monitored by silica gel TLC in toluene:acetone (2:1, v/v) solvent system (R_{f tax.}=0.38, R_{f prod}.=0.64), and when formation of higher mobility products was observed the reaction was quenched by evaporation of solvents using Rotavapor at 30. degree. C. A TLC analysis of the reaction mixture indicated the presence of some quantities of unreacted paclitaxel and 2′,7-O-bis(methylthiomethyl)paclitaxel. Separation of the title compound from the reaction mixture was achieved by flash column chromatography on Silica Gel 60 (40-63 .mu.m) EM Science (100 mL), column diameter: 2 in. using ethyl acetate:hexane (1:1, v/v) solvent system (R_{f prod}.=0.34). The product (552 mg, 60% yield) was recovered from fractions 12 to 18 (each fraction ca. 20 ml).

MS (FAB/matrix NOBA, NaI, KI): [M+H].sup.+, m/z 914; [M+Na].sup.+, m/z 936; [M+K].sup.+, m/z 952. Elemental Analysis: C: 64.28 (calc. 64.39), H: 5.85 (calc. 6.07), N: 1.46 (calc. 1.53). UV (MeOH): .λ.max=226 nm, E(1%/1 cm)=150, A=0.2653. IR (KBr): 3432, 3066, 2940, 1726, 1668, 1602, 1582, 1514, 1484, 1452, 1372, 1242, 1178, 1142, 1108, 1068, 1026, 990, 916, 884, 852, 802, 774, 710, 608, 570, 538, 482 cm^{-1.} .¹H-NMR (CDCl₃) δ 1.15 (3H, s), 1.19 (3H, s), 1.73 (3H, s), 1.79 (H, s), 1.90 (3H, d), 2.09 (3H, S), 2.16 (3H, s), 2.29 (2H, d), 2.35 (3H, s), 2.77 (H, m), 3.70 (H, d), 3.83 (H, d), 4.17 (H, d), 4.26 (H, m, overlaps with H, d), 4.63 (2H, t), 4.77 (H, dd), 4.91 (H, d), 5.65 (H, d), 5.77 (H, dd), 6.16 (H,dd), 6.48 (H, s), 7.07 (H, d), 7.29-7.50 (10H, m), 7.57 (H, m), 7.73 (2H, d), 8.08 (2H, d).

The present invention also contemplates kits, for example, for inhibiting tumor growth comprising a first container (such as a vial) containing a pharmaceutical formulation comprising a compound of the present invention, said compound in a pharmaceutically acceptable carrier, and a second container (such as a vial) containing a co-solvent to be used in combination with said compound of the present invention, the contents of said containers being mixed prior to administration.

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

We claim:

1. A pharmaceutical composition for administration to a patient which comprises in a first container
a) a pharmaceutically effective amount of at least one compound of the formula 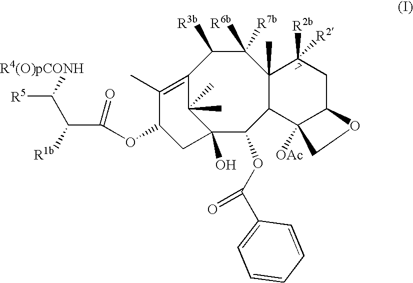
wherein:
R^{1b} is hydroxy, protected hydroxy, —OCH₂SCH₃, —OC(O)R^{x} or —OC(O)OR^{x};
R² is hydrogen, and
R^{2b} is hydrogen, hydroxy, protected hydroxy, —OCH₂SCH₃ or —OC(O)OR^{x};
R^{3b} is hydrogen, hydroxy, protected hydroxy, C₁₋₆ alkyloxy, —OC(O)R^{x}, —OCH₂SCH₃ or —OC(O)OR^{x};
one of R^{6b} or R^{7b} is hydrogen and the other is hydroxy, protected hydroxy, C₁₋₆ alkanoyloxy or —OCH₂SCH₃; or
R^{6b} and R^{7b} together form an oxo group; with the proviso that at least one of R^{1b}, R^{2b}, R^{3b}, R^{6b} or R^{7b} is —OCH₂SCH₃;
p is 0 or 1;
R^{x} is a radical of the formula 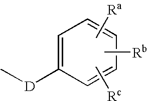
wherein
D is a bond or C₁₋₆ alkyl; and
R^{a}, R^{b} and R^{c} are independently hydrogen, amino C₁₋₆ alkylamino, di-C₁₋₆ alkylamino, halogen, C₁₋₆ alkyl, or C₁₋₆ alkoxy;
R⁴ and R⁵ are independently C₁₋₆ alkyl, C₂₋₆ alkenyl, C₂₋₆alkynyl, or —ZR⁶; wherein Z is a direct bond, C₁₋₆ alkyl or C₂₋₆ alkenyl; and
R⁶ is aryl, substituted aryl, C₃₋₆ cycloalkyl, or heteroaryl;
b) in a suitable solvent; and
c) a pharmaceutically effective amount of a buffer;
and in a second container;
d) a pharmaceutically effective amount of a co-solvent; and
e) a pharmaceutically effective amount of a buffer.

2. The composition of claim 1 wherein the contents of the first container and the contents of the second container are mixed just prior to administration.

3. The composition in accordance with claim 1, which comprises in a first container
a) a pharmaceutically effective amount of a compound of the formula 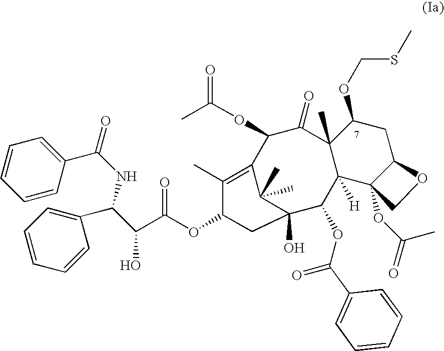
b) in a suitable solvent; and
c) a pharmaceutically effective amount of a buffer; and
in a second container;
d) a pharmaceutically effective amount of a co-solvent; and
e) a pharmaceutically effective amount of a buffer.

4. The composition of claim 3 wherein the contents of the first container and the contents of the second container are mixed just prior to administration.

5. The composition of claim 1 wherein the suitable solvent in step (b) is selected from ethanol, t-butyl alcohol, propylene glycol, glycerin, benzyl benzoate and N,N-dimethylacetamide.

6. The composition of claim 1 wherein the buffer in step (c) is a citrate, tartrate, succinate, fumarate, oxalate, benzoate, acetate or lactate buffer.

7. The composition of claim 1 wherein the co-solvent in step d) is polyoxyethylated (POE) castor oil, polysorbate or polyethylene glycol.

8. The composition of claim 1 wherein the buffer in step e) is a tartrate buffer.

9. The composition of claim 1 which comprises in the first container about 1 μg/mL to about 20 mg/mL of Compound I, about 0.05 to about 0.95 mL/mL ethanol in an aqueous tartrate buffer, and in the second vial about 0.01 to about 0.95 mL/mL of a polyoxyethylated castor oil in an aqueous tartrate buffer.

10. The composition of claim 9 wherein the compound of the formula 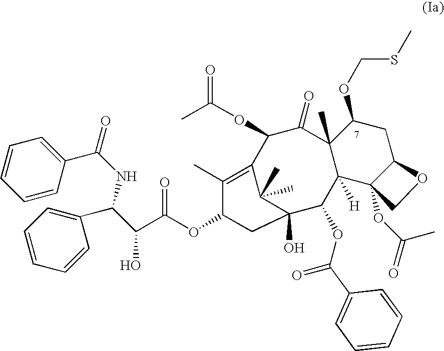
is employed in the first container.

11. The composition of claim 10 which comprises in the first container 15.0 mg/mL of Compound Ia, .0.75 mL/mL of dehydrated alcohol, 0.22 mg/mL of tartaric acid, 0.31 mg/mL of sodium tartrate dihydrate and water, and in the second container, 0.044 mL/mL of POE castor oil, 0.086 mg/mL of tartaric acid, 2.07 mg/mL of sodium tartrate dihydrate and water.

12. A process for preparing a pharmaceutical composition which comprises mixing a compound of the formula 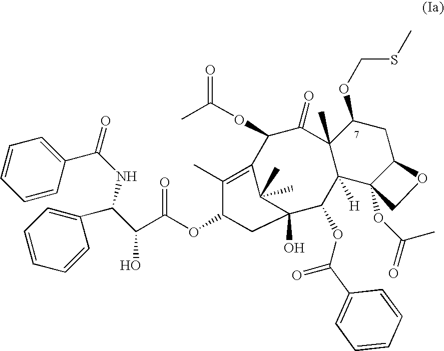
in solution with 75% v/v ethanol:aqueous tartrate buffer with a solution of polyoxyethylated castor oil in an aqueous tartrate buffer prior to administration to a patient.

13. The process of claim 12 wherein the composition is administered intravenously.

14. The pharmaceutical composition of claim 1 wherein the composition comprises an antitumor effective amount of the compound of the formula 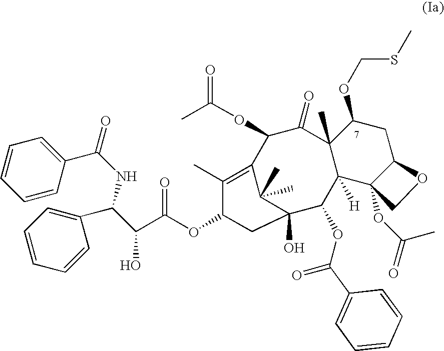
in a pharmaceutically acceptable carrier.

15. A method for inhibiting tumor growth which comprises administering to a patient in need thereof a tumor-growth inhibiting amount of the composition as claimed in claim 1.

16. A kit for inhibiting tumor growth which comprises a first container containing a pharmaceutical formulation comprising a compound of claim 1, said compound in a pharmaceutically acceptable carrier, and a second container containing a co-solvent to be used in combination with a compound of claim 1, the contents of said containers being mixed prior to administration.
